(19) United States
(12) Patent Application Publication
(10) Pub. No.: US 2008/0186480 A1
(43) Pub. Date: Aug. 7, 2008

(54) Apparatus, System and Method of Controlling and Monitoring the Energy of a Laser

(76) Inventors: Stefan Lang, Markt Schwaben (DE); Roland Toennies, Olching (DE)

Correspondence Address: Bausch & Lomb Incorporated, One Bausch & Lomb Place, Rochester, NY, 14604-2701, US

(21) Appl. No.: 12/063,901

(22) PCT Filed: Sep. 27, 2006
(86) PCT No.: PCT/EP2006/009395

§ 371 Date: Feb. 15, 2008

(30) Foreign Application Priority Data
Sep. 27, 2005 (DE) 10 2005 046 129.8

(51) Int. Cl. G01J 1/02; A61B 5/00

(52) U.S. Cl. 356/213

(57) ABSTRACT

The present invention is based on the concept to detect the noise which is generated when a laser pulse of the excimer laser hits on a reference material. In particular where the laser pulse of an excimer laser hits on a reference material the radiation ablates a corresponding volume of the reference material by photodecomposition. The ablated volume of material which is proportional to the pulse energy applied to the reference material can be determined based on measuring the acoustic shock wave resulting from the ablation. The reference material is preferably a plate made of a material erodable by an excimer laser, more preferably a plate made of plastics and most preferably PMMA.

### FIELD OF THE INVENTION

The present invention relates to an apparatus, system and a method of controlling and monitoring the energy of a laser, in particular to an apparatus and a method for monitoring the energy of an excimer laser for use in a refractive laser system.

### DESCRIPTION OF THE RELATED ART

U.S. Pat. No. 6,195,164 B1 relates to a system and method for calibrating laser ablations. This known method is based on measuring the optical power and shape of a test surface that has been ablated by energy delivered from a laser. The interaction of a geometrical pattern superimposed with the ablation test surface is analysed using a microscope, video camera connector and other existing components of a laser ablation system. If desired, the known optical properties of the ablated test surface may be used to adjust the laser ablation system by varying treatment parameters such as laser pulse intensity and exposure time.

### SUMMARY OF THE INVENTION

The object underlying the present invention is to provide an apparatus and a method for monitoring the energy of a laser.

This object is solved with the features of the claims.

The present invention is based on the concept to detect the noise which is generated when a laser pulse of the excimer laser hits on a reference material. In particular where the laser pulse of an excimer laser hits on a reference material the radiation ablates a corresponding volume of the reference material by photodecomposition. The ablated volume of material which is proportional to the pulse energy applied to the reference material can be determined based on measuring the acoustic shock wave resulting from the ablation. The reference material is preferably a plate made of a material erodable by an excimer laser, more preferably a plate made of plastics and most preferably PMMA.

An apparatus according to the present invention comprises a microphone, which provides an electrical signal, when a laser pulse hits on the reference material. The electrical signal corresponds to the pressure of the shock wave which propagates from the position where the laser pulse hits on the reference surface to the microphone.

The electrical signal from the microphone is provided to a processing means which receives said electrical signal and generates a reference data which is a measure of the energy of the laser pulse and correspondingly a measure of the ablation rate and/or the size of the ablation area.

According to a preferred embodiment of the present invention, the processing means comprises an amplifier which receives the electrical signal of the microphone and amplifies the signal for further processing. Preferably, the output signal of the amplifier is converted into a digital signal by using an analog-to-digital converter. The digital signal is then provided to a digital analyser, preferably a microprocessor or a microcomputer.

A typical electrical signal provided by the microphone has a form like an attenuated sinusoidal signal. Thus starting from a base which represents the back noise the amplitude of the electrical signal becomes smaller over time and reaches a specific minimum Eₘᵢₙ₁ at a corresponding time tₘᵢₙ₁. The amplitude then becomes larger again up to a first signal maximum Eₘₐₓ₁ at a corresponding time tₘₐₓ₁. The signal further changes to a second minimum Eₘᵢₙ₂ and thereafter to a second maximum Eₘₐₓ₂ and so on. The absolute value of the second minimum Eₘᵢₙ₂ is smaller than the absolute value of the first minimum Eₘᵢₙ₁ and similarly the absolute value of the second maximum Eₘₐₓ₂ is smaller than the absolute value of the first maximum Eₘₐₓ₁.

According to a preferred embodiment of the present invention the value of the amplitude at the first signal minimum Eₘᵢₙ₁ is used for determining a measure of the pressure amplitude of the shock wave. For evaluating the amplitude preferably three parameters are taken, i.e. the value for the base signal, i.e. the background noise signal which preferably is an average over ten samples. The second parameter is a peak value, i.e. the digital value of the first minimum Eₘᵢₙ₁. The third parameter is the position of the first minimum Eₘᵢₙ₁, i.e. the point in time tₘᵢₙ₁ with reference to a starting time to when the laser pulse hits the reference surface or with reference to the time when a trigger signal is sent to the laser system.

The signal amplitude is determined as the difference between the value of the base signal and the peak value.

The present invention provides a method of controlling and monitoring the energy of laser pulses in particular of an excimer laser. This method comprises a calibration routine, an adjustment routine and a monitoring routine.

According to a further aspect of the invention, every n-th laser pulse from a series of laser pulses is directed to a defined position on the reference material. The number n is a natural number greater than 2, preferably 25 to 200, more preferably 100. Depending on the pulse rate of the laser system an appropriate number n is chosen. According to this preferred embodiment the corresponding electrical signal of said n-th laser pulse is evaluated. This has the advantage that the processing means for evaluating the electrical signal can be simplified while the laser system is tested under normal operating condition, i.e. at a high pulse rate, for example 500 Hz. The other laser pulses from said series of laser pulses are directed to a park position on the reference material or into a beam dump. This has the further advantages that by applying only every n-th laser pulse of said series of laser pulses to the measurement position at the reference material one can avoid in the case of using plastics that the material is heated which may result in a carbonisation of the material. Moreover, upon ablation of material the ablated material may form a cloud around the measurement position of the reference material. If there is sufficient time between subsequent laser pulses hitting on the measurement position at the reference material this cloud will disappear so that the following pulse is not effected by this cloud of debris.

### BRIEF DESCRIPTION OF THE DRAWINGS

The invention will be further described by way of examples with reference to the drawings, in which:

FIG. 1 is a schematic diagram illustrating the apparatus according to a preferred embodiment of the present invention;

FIG. 2 is a diagram showing the output signal of a microphone;

FIG. 3 shows a diagram of the energy distribution of a laser beam;

FIG. 4 schematically shows a panel with a display for controlling and monitoring the laser energy;

FIG. 5 shows a flow chart for an automatic energy adjustment using the present invention and

FIG. 6 shows a schematic diagram when performing the method according to the present invention.

### DETAILED DESCRIPTION OF THE PREFERRED EMBODIMENTS

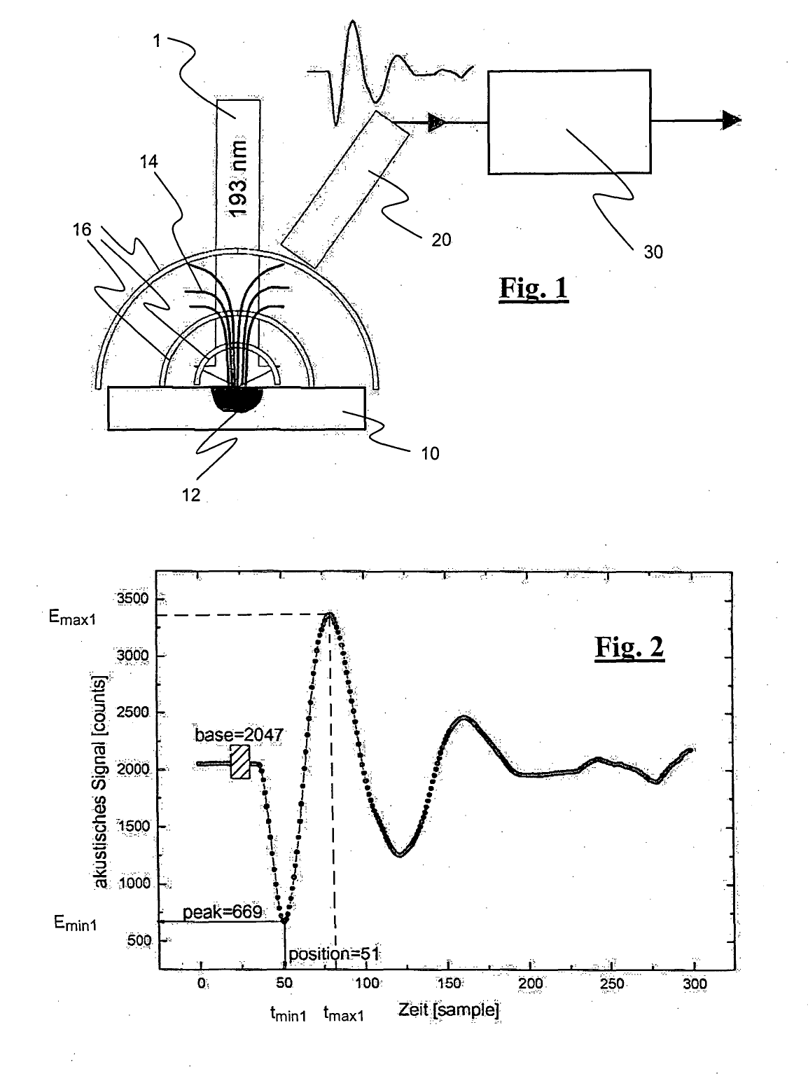

FIG. 1 shows a schematic diagram illustrating the apparatus according to a preferred embodiment to the present invention. The arrangement comprises a reference material 10 which may be a plate of a suitable test material, preferably polycarbonate and more preferably PMMA. Any reference material can be used, where the application of a laser pulse on the test surface creates an acoustical effect. More preferably, any material can be used which upon ablation by a laser pulse of an excimer laser preferably working at a wave length of 193 nm results in an acoustical shock wave. The apparatus further comprises a detector for detecting the acoustical sound and for providing an electrical signal. In the present embodiment a microphone 20 is used which converts the pressure of the acoustical shock wave into an electrical signal. An output of the microphone 20 is connected to a processing means 30. The processing means analyses the electrical signal received from the microphone and outputs data being a measure of the electrical signal to a personal computer (PC).

FIG. 1 further shows in a diagrammatic form that a laser pulse 1 hits the upper surface of the reference material 10 at a measurement position 12. As diagrammatically shown at the measurement position 12 material has been ablated which spreads around as indicated by lines 14. Reference number 16 indicates the acoustic sound which propagates away from the measurement position 12.

In FIG. 2 a diagram is shown of an example of an output signal of a microphone 20 which is processed in the processing means 30. It shows the amplitude of the acoustical signal with a unit of counts which changes over the time. The time is shown with the unit of samples taken during the measurement. In a preferred embodiment the sampling rate for taking the samples is 1.2 MHz.

More specifically, FIG. 2 shows the signal starting with a base signal followed by an attenuated sinusoidal signal. In this example, the base signal representing the background noise is taken as an average over 10 samples. The base value in this example is 2047. A first signal minimum Eₘᵢₙ₁ has a peak value of 669. It corresponds to the sample at position 51 corresponding to a time tₘᵢₙ₁. According to a preferred embodiment of the present invention these three evaluation parameters, i.e. the base value, the peak value and the position value are outputted to the personal computer for further processing.

The signal form as shown in FIG. 2 further comprises a first signal maximum Eₘₐₓ₁ at a position tₘₐₓ₁ followed by a second signal minimum Eₘᵢₙ₂ and thereafter a second signal maximum Eₘₐₓ₂ at corresponding time tₘᵢₙ₂ and tₘₐₓ₂.

In the present preferred embodiment the acoustical signal amplitude corresponds to the difference between the base value and the peak value. However, further information can be used for evaluating the acoustical shock wave which corresponds to the laser energy and the laser size as well as laser form of any laser pulse hitting the reference material. For example, the first signal maximum and any further signal minima and signal maxima can be used for evaluation. Moreover, the point in time of the respective maxima and minima can be used for the evaluation.

A measurement will be performed as follows. A test plate made of polycarbonate (PC) is positioned at the same level or hight as the treatment surface next to but spaced apart from said measurement position. When using a laser having a repetition rate of 500 Hz the energy check may be performed by measuring every one out of 100 laser pulses, i.e. n=100 that means every 100-th laser pulse is evaluated (measurement frequency 5 Hz). During this measurement one laser pulse is directed to a measurement position (0.0) whereas other 99 laser pulses are directed to a park position (0, −12500).

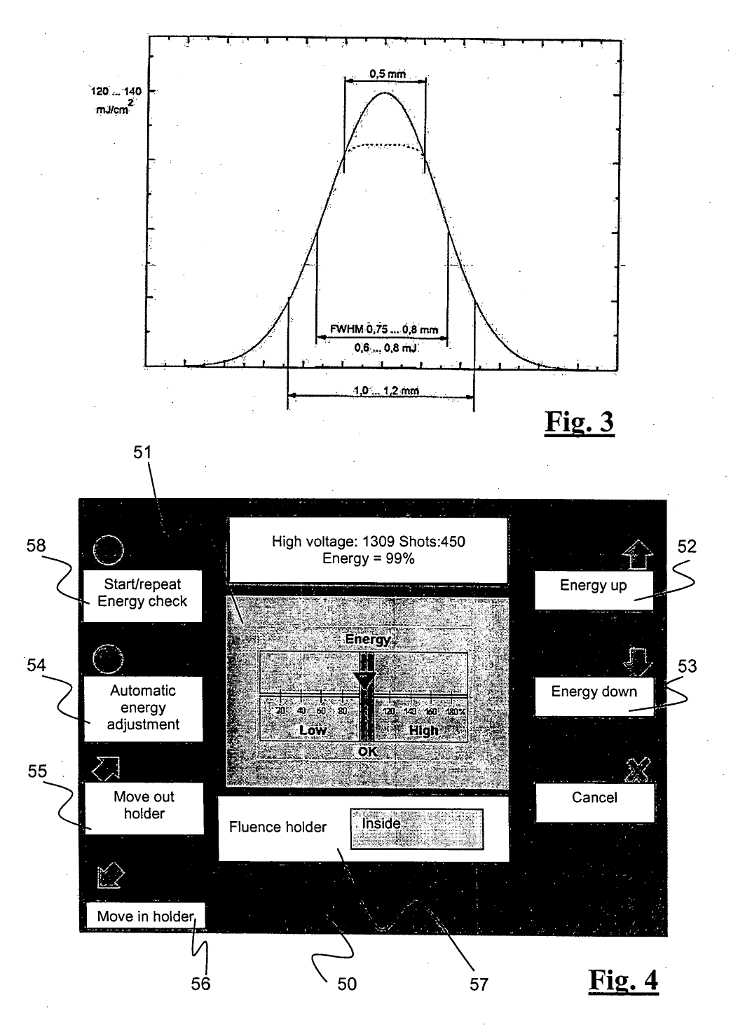

In FIG. 3 a diagram of the energy distribution of an exemplary laser beam is shown. More specifically, it shows the energy versus width of the laser spot. In this example the maximum is about 120-140 mJ/cm². The value of FWHM (full width at half maximum) is about 0.75 to 0.8 mm at the level of the treatment surface. The aspect ratio is better than 1:1.1. During the calibration routine the target energy, the target size and the target shape is adjusted so that the target laser spot at the treatment level is obtained. Then the corresponding acoustical signal when ablating a reference surface made of polycarbonate with this target spot is stored as the target value corresponding to 100%.

FIG. 4 shows a panel 50 with a display 51 for controlling and monitoring the laser energy. The display comprises a scale showing values from 20 to 180%. The region of 100%±5% is shown as a vertical beam wherein a triangle points to the actual value. As long as the actual value is within this region of 100%±5% the energy check is taken to be successful. However, if the energy is to low or to high the laser energy may be varied by varying the high voltage of the laser. This can be done by using the buttons 52, 53 “energy up” or “energy down”. The user may also select the button for “automatic energy adjustment” 54. The panel further comprises buttons 55, 56 for “moving in” and “moving out” a holder 57 for the reference material, i.e. the test plate.

According to a preferred embodiment measurement is performed based on 50 measurement pulses (which corresponds to 5000 laser pulses). During this measurement the high voltage of laser is kept unchanged. After the user has manually changed the high voltage a new energy check is performed by pressing a button 58.

When using automatic energy adjustment the laser software adjusts a high voltage of the laser until reaching the target value. This is usually achieved after 150 measurement pulses. Upon a successful energy check the signal of a photonic energy monitor is stored as a reference value for the treatment.

At every measurement pulse the data provided by the acoustical energy monitor is used for calculating the acoustical signal in percent and then the corresponding value is displayed. The average value of the acoustical signals are shown in the graphical representation in percent. At the end of the energy check the average value of the acoustical signals is outputted.

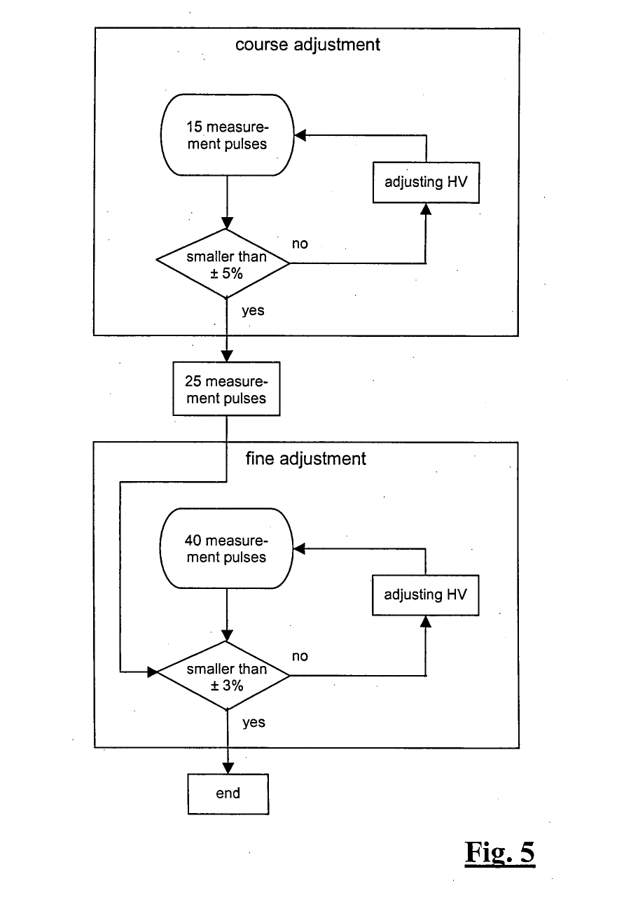

With reference to FIG. 5 a flow chart for an automatic energy adjustment will be described. The automatic energy adjustment is preferably done in several adjustment cycles until the difference between the actual energy and the target energy is less than ±3%.

After pressing the button “automatic energy adjustment” the software as well as the acoustical and photonic energy monitor is put into an initial stage. Upon pressing a foot switch 150 measurement pulses (at maximum) are directed to the test specimen. After 15 measurement pulses a check is performed for a course adjustment wherein the high voltage of the laser is adjusted if the difference between the actual energy and the target energy is larger than ±5%. After further 15 measurement pulses the corresponding average of the energy is checked again and if necessary a further adjustment of the high voltage is performed. When the criteria of this course adjustment is achieved further 25 measurement pulses are applied to the test specimen. Than it is checked whether the average value of the energy fulfils the fine adjustment criteria of ±3%. If it does not fulfil this criteria the energy of the laser is adjusted. After further 40 measurement pulses it is again checked whether the fine adjustment criteria is fulfilled. As soon as the criteria is fulfilled the program goes to the end of the adjustment procedure. Thereafter, the treatment with the laser system will be allowed within a predetermined time. This predetermined time may be selected by the user and can be for example any time between two minutes and 20 minutes.

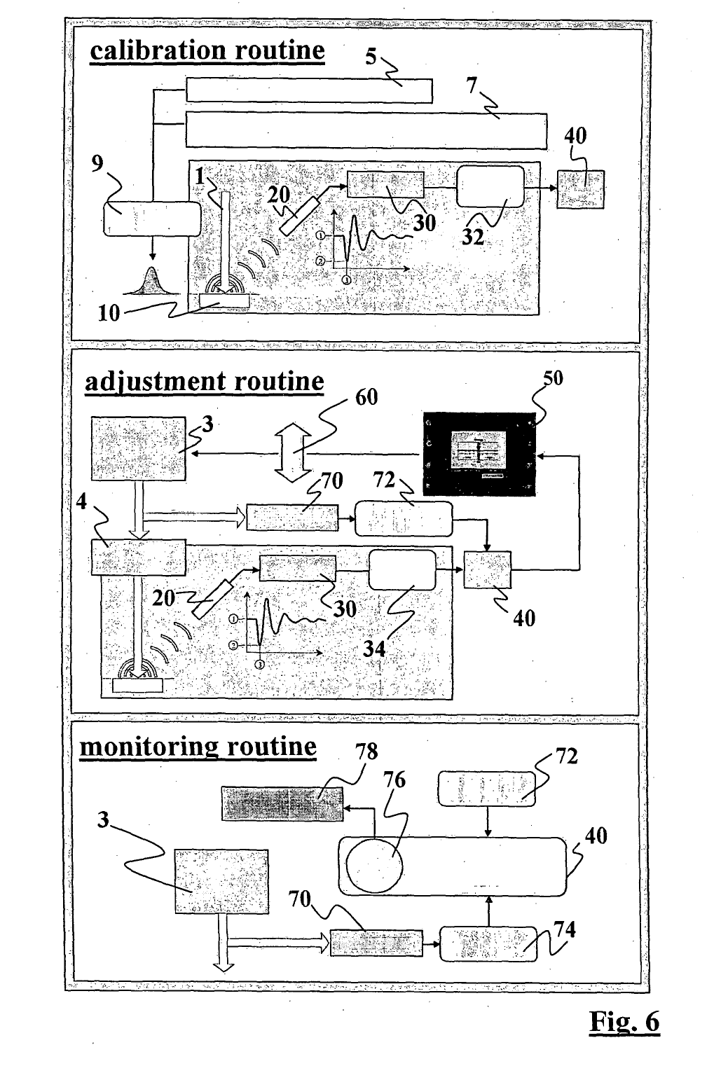

In FIG. 6 a schematic diagram for performing a calibration routine, an adjustment routine and a monitoring routine is shown.

The calibration routine is preferably performed during the service before delivering the laser system to a user and thereafter at regular intervals for checking the functioning of the laser system. More specifically, in a test environment the laser system is used for providing a laser pulse 1 to a test material 10 which is positioned at the treatment position, i.e. at the same place and hight where treatment of a patient's eye is performed. The laser system is adjusted in such a way that the laser pulse 1 hitting on the test material 10 provides the target energy which is measured by an appropriate system for example by using a joule meter 5 for measuring the energy and for measuring the power, respectively. As such a joule meter preferably a molectron EPM-1000 in combination with the measuring head J8-LP4 or PB-10X is used. This known apparatus uses a measurement principle wherein the pulse energy or the average power is determined by using a pyrroelectric or thermo measurement head. Preferably, the measurement is performed at the treatment position but alternatively any arbitrary position in the system may be used.

The laser system is further adjusted such that a target laser pulse 9 which hits on the test material in the treatment surface has a predetermined target energy distribution, a predetermined target shape and a predetermined target size (target diameter). This measurement can be performed by appropriate apparatuses 7 for example a beam profiler. For this measurement preferably a test surface comprising fluorescent material is used. The beam profiler 7 preferably comprises a CCD-camera (charged a coupled device) comprising a camera chip for detecting any fluorescence when laser pulses hit on the fluorescent test surface of the beam profiler. Alternatively, a profilometer can be used for determining the profile of an ablated material in a test surface. More preferably a test material comprising a plastics material made of polycarbonate (PC) or alternatively PMMA is used. By using a laser profilometer or a p-SCAN-device the ablated volume of the material is measured.

For checking the energy distribution and the shape and size further tests may be performed, for example refraction tests.

With a laser system adjusted according to the target energy including the before mentioned parameters an acoustical sensor 20, 30 according to the present invention is used for measuring the acoustic shock wave resulting from the ablated volume of material when using laser pulses of said laser system. More specifically, the laser beam is directed to a reference material and noise being created when a laser pulse hits on the reference material is received by a microphone 20 which provides a signal to the processing means 30. The processing means 30 provides preferably three parameter values 32 comprising the base value, the peak value and the position value. These signals are provided in this example to a personal computer 40 of the laser system as the target values for later use. In the present example these target values are each related to 100%. After this calibration routine which may be repeated as discussed above at regular intervals the laser system will be used as described in the following.

Before a treatment of a patient's eye is performed the user may check the energy of the laser pulse by way of the adjustment routine. The beam 1 of the excimer laser 3 is directed via an optical system 4 to the reference material 10 and the noise of the acoustical shock wave is measured. The processing means 30 provides the information regarding the actual value of the energy. In the present example the measured parameters 34 are the actual base value, the actual peak value and the actual position value. These values are provided to the personal computer 40 of the laser system. In the personal computer each of the actual values 34 are compared with each of the respective target values 32. The result of this comparison is provided to a display 50.

During performing the energy check the actual value provided by the acoustical sensor may deviate from the target value by ±5% of the target value which is taken as a 100% value.

The user may then manually change the energy of the excimer laser for example by reducing or increasing the high voltage for the laser 3. Preferably, the result of this comparison is used for automatic adjustment 60 of the energy of the laser for example for automatically reducing or automatically increasing the high voltage of the laser.

The laser system preferably comprises a photonic energy monitoring means 70 for measuring the laser energy during the treatment. Preferably, a part of the laser beam for example by using a partly reflecting mirror is guided to the photonic energy monitoring means 70. According to the present invention the photonic energy monitoring means provides a reference value 72 representing the energy value of the laser beam to the personal computer 40. This reference value 72 is taken at the same time when performing the energy check by the user or performing the automatic energy check. This reference value 72 of the photonic energy monitoring means 70 is used for monitoring the actual energy during a treatment.

During performing a treatment of a patient's eye a monitoring routine is performed. The photonic energy monitoring means 70 is continuously delivering the actual value 74 to the personal computer 40. The personal computer 40 performs a comparison of the actual value 74 with the reference value 72 previously stored therein. If the difference 76 between the actual value 74 and the reference value 72 becomes greater than a predetermined value the personal computer 40 provides a command signal 78 to the laser system for stopping the laser treatment. In an example the treatment is stopped when the difference 76 between the actual value 74 and the reference value 72 amounts to 2.5% of the reference value. Thus, if the actual energy of the laser beam decreases or increases so that the difference becomes larger than 2.5% of the reference value the treatment is stopped.

According to the present invention the reference value 72 taken with the photonic energy monitoring means is an average value for the last 300 pulses during the energy check of the adjustment routine. Correspondingly, the actual value 74 provided by the photonic energy monitoring means is an average value taken over 300 pulses during the treatment.

The foregoing disclosure and description of the invention are illustrative and explanatory thereof, and whereas changes in the size, shape, materials, components, circuit elements, wiring connections and contacts, as well as in the details of the illustrated apparatus and construction and method of operation may be made without departing from the scope of the invention.

1. A laser system comprising:
an excimer laser;
means for directing a laser pulse of the excimer laser to a measurement position at a reference material;
an apparatus for measuring the energy of the laser pulse of the excimer laser hitting on the measurement position on the reference material comprising means to detect the noise; and
means for selecting every n-th laser pulse from a series of laser pulses to be directed to the measurement position at the reference material a natural number greater than 2.

2. Laser system of claim 1, wherein the noise detector is adapted to measure the acoustic shock wave resulting from ablation of a volume of the reference material corresponding to the energy of the laser pulse.

3. Laser system of claim 1 wherein the noise detector comprises a microphone which provides an electrical signal corresponding to the pressure of a shock wave which propagates from a position where the laser pulse hits on the reference material.

4. Laser system of claim 3, further comprising a processing means which receives said electrical signal from the microphone for generating a reference data which is a measure of the energy of the laser pulse hitting on the reference material.

5. Laser system of claim 4, wherein the processing means comprises an amplifier receiving the electrical signal of the microphone for amplifying the signal, an analog-to-digital converter for converting said amplified signal into a digital signal and a digital analyser receiving said digital signal.

6. Laser system of claim 4 wherein said processing means is adapted to provide three parameter values comprising a base value representing the background noise, a peak value and the corresponding position value of a first minimum Eₘᵢₙ₁ of said electrical signal as a measure of the detected noise.

7. Laser system of claim 6, further comprising a personal computer for receiving said three parameter values as actual values for a laser pulse of the excimer laser hitting on the reference material and for comparing said actual values with target values previously stored for a laser pulse of a calibrated laser, and said personal computer provides a result of said comparison.

8. Laser system of claim 7, further comprising means for automatic adjustment of the energy of the laser receiving a control signal corresponding to a result of the comparison provided by the personal computer by automatically reducing or automatically increasing the high voltage of the laser corresponding to said control signal.

9. Laser system of claim 1 wherein said number n is 25 to 200.

10. Laser system of claim 1 further comprising a photonic energy monitoring means and a split mirror for directing a part of the laser beam to the photonic energy monitoring means.

11. Laser system of claim 10, wherein said personal computer is adapted to receive an actual value of said photonic energy monitoring means for performing a comparison of the actual value with a reference value previously stored in said personal computer.

12. Laser system of claim 11, wherein said photonic energy monitoring means comprises means for generating an average value over 300 pulses of said excimer laser.

13. Method for measuring the energy of a laser pulse of an excimer laser hitting on a reference material comprising the step of (a) directing a laser pulse of the excimer laser to a measurement position at the reference material or another position, preferably a park position on said reference material; (b) detecting the noise at the measurement position; and (c) selecting every n-th laser pulse from a series of laser pulses to be directed to the measurement position at the reference material, wherein said number is a natural number greater than 2.

14. Method of claim 13 wherein, the step of detecting the noise comprises measuring the acoustic shock wave resulting from ablation of a volume of the reference material corresponding to the energy of the laser pulse.

15. Method of claim 13 wherein the step of detecting the noise comprises providing an electrical signal corresponding to the pressure of a shock wave which propagates from a position where the laser pulse hits on the reference material.

16. Method of claim 15, further comprising the step of processing said electrical signal for generating a reference data which is a measure of the energy of the laser pulse hitting on the reference material.

17. Method of claim 16, wherein the processing step comprises amplifying the electrical signal analog-to-digital converting said amplified signal into a digital signal and digital analysing said digital signal.

18. Method of claim 16 wherein said processing step comprises providing three parameter values, a base value representing the background noise, a peak value and the corresponding position value of a first minimum Eₘᵢₙ₁ of said electrical signal as a measure of the detected noise.

19. Method of claim 18, further comprising comparing said three parameter values as actual values for a laser pulse of the excimer laser hitting on the reference material with target values previously stored for a laser pulse of a calibrated laser, and providing a result of said comparison.

20. Method of claim 19, further comprising providing a control signal corresponding to a result of the comparison for automatic adjustment of the energy of the laser by automatically reducing or automatically increasing the high voltage of the laser corresponding to said control signal.

21. Method of claim 13 wherein said number n is 25 to 200.

22. Method of claim 13 further comprising the step of photonic energy monitoring using a photonic energy monitoring means and a split mirror for directing a part of the laser beam to the photonic energy monitoring means.

23. Method of claim 22, further comprising comparing an actual value of said photonic energy monitoring means with a reference value previously stored.

24. Method of claim 23 wherein the step of photonic energy monitoring comprises generating an average value over 300 pulses of said excimer laser.

25. Laser system of claim 2 wherein the noise detector comprises a microphone which provides an electrical signal corresponding to the pressure of a shock wave which propagates from a position where the laser pulse hits on the reference material.

26. Laser system of claim 25 further comprising a processing means which receives said electrical signal from the microphone for generating a reference data which is a measure of the energy of the laser pulse hitting on the reference material.

27. Laser system of claim 26 wherein the processing means comprises an amplifier receiving the electrical signal of the microphone for amplifying the signal, an analog-to-digital converter for converting said amplified signal into a digital signal and a digital analyser receiving said digital signal.

28. Laser system of claim 5 wherein said processing means is adapted to provide three parameter values comprising a base value representing the background noise, a peak value and the corresponding position value of a first minimum Eₘᵢₙ₁ of said electrical signal as a measure of the detected noise.

29. Laser system of claim 26 wherein said processing means is adapted to provide three parameter values comprising a base value representing the background noise, a peak value and the corresponding position value of a first minimum Eₘᵢₙ₁ of said electrical signal as a measure of the detected noise.

30. Laser system of claim 27 wherein said processing means is adapted to provide three parameter values comprising a base value representing the background noise, a peak value and the corresponding position value of a first minimum Eₘᵢₙ₁ of said electrical signal as a measure of the detected noise.

31. Laser system of claim 28 further comprising a personal computer for receiving said three parameter values as actual values for a laser pulse of the excimer laser hitting on the reference material and for comparing said actual values with target values previously stored for a laser pulse of a calibrated laser, and said personal computer provides a result of said comparison.

32. Laser system of claim 31 further comprising means for automatic adjustment of the energy of the laser receiving a control signal corresponding to a result of the comparison provided by the personal computer by automatically reducing or automatically increasing the high voltage of the laser corresponding to said control signal.

33. Method of claim 14 wherein the step of detecting the noise comprises providing an electrical signal corresponding to the pressure of a shock wave which propagates from a position where the laser pulse hits on the reference material.

34. Method of claim 33 further comprising the step of processing said electrical signal for generating a reference data which is a measure of the energy of the laser pulse hitting on the reference material.

35. Method of claim 34 wherein the processing step comprises amplifying the electrical signal analog-to-digital converting said amplified signal into a digital signal and digital analysing said digital signal.

36. Method of claim 17, wherein said processing step comprises providing three parameter values, a base value representing the background noise, a peak value and the corresponding position value of a first minimum Eₘᵢₙ₁ of said electrical signal as a measure of the detected noise.

37. Method of claim 33 wherein said processing step comprises providing three parameter values, a base value representing the background noise, a peak value and the corresponding position value of a first minimum Eₘᵢₙ₁ of said electrical signal as a measure of the detected noise.

38. Method of claim 34 wherein said processing step comprises providing three parameter values, a base value representing the background noise, a peak value and the corresponding position value of a first minimum Eₘᵢₙ₁ of said electrical signal as a measure of the detected noise.

39. Method of claim 36 further comprising comparing said three parameter values as actual values for a laser pulse of the excimer laser hitting on the reference material with target values previously stored for a laser pulse of a calibrated laser, and providing a result of said comparison.

40. Method of claim 39 further comprising providing a control signal corresponding to a result of the comparison for automatic adjustment of the energy of the laser by automatically reducing or automatically increasing the high voltage of the laser corresponding to said control signal.

41. Laser system of claim 1 wherein the laser pulse of the excimer laser is directed to a park position on said reference material.
